(19) United States
(12) Patent Application Publication
(10) Pub. No.: US 2025/0020630 A1
(43) Pub. Date: Jan. 16, 2025

(54) ANALYTE MONITORING METHODS AND SYSTEMS

(71) Applicant: Intuity Medical, Inc., Fremont, CA (US)

(72) Inventors: Kelley J. LIPMAN, Livermore, CA (US); Robin S. GAFFNEY, Redwood City, CA (US); Kimberly J. TANSEY, San Carlos, CA (US)

(21) Appl. No.: 18/444,330

(22) Filed: Feb. 16, 2024

Related U.S. Application Data
(63) Continuation of application No. 16/402,120, filed on May 2, 2019, now abandoned.
Application No. 16/402,120 is a continuation of application No. 13/168,644, filed on Jun. 24, 2011, now Pat. No. 10,330,667.
(60) Provisional application No. 61/358,825, filed on Jun. 25, 2010.

(51) Int. Cl. G01N 33/487; A61B 5/00; A61B 5/145; A61B 5/15; A61B 5/151; A61B 5/157; G16H 20/10; G16H 20/60; G16H 40/63

(52) U.S. Cl. CPC G01N 33/48792; A61B 5/150022; A61B 5/150358; A61B 5/157; G16H 40/63; A61B 5/14532; A61B 5/151; A61B 5/15186; A61B 5/7475; G16H 20/10; G16H 20/60

(57) ABSTRACT

A method is disclosed involving monitoring the concentration of at least one target analyte in a sample of body fluid using a meter, the meter including a user interface, the method including: obtaining a sample of body fluid; testing the sample to determine the concentration of the at least one target analyte contained therein; and presenting the user with a reminder to associate the test with an appropriate time corresponding to before or after a particular meal using the user interface. Associated devices, systems and arrangements are also disclosed.

### FIELD

The invention described herein relates to methods, devices, arrangements and/or systems for monitoring a target analyte in a simpler, more accurate manner.

### BACKGROUND

In this specification where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

Currently, commercially available glucose monitoring systems typically contain a number of separate components. Namely, separate lancing devices for creating a droplet of blood on the skin, test strips, and a meter configured to receive one test strip at a time. A test strip is inserted into the meter before or after the skin is lanced with the separate lancing device, then the strip is maneuvered into contact with the droplet of blood on the surface of the skin. The strip absorbs the blood, then the blood is analyzed by the strip/meter to determine the concentration of glucose contained therein.

Many of these devices allow people with diabetes to mark an individual glucose measurement to associate the result with a particular meal, or meal-time. For example, the meter might allow a user to, after a result is displayed on the meter, associate that result with “breakfast”. If a user diligently marked their results associating them with the appropriate meal-time, then a user's health care provider may be able to analyze the data looking for trends in measurement that can be used to adjust the delivery time and dosage of any treatments provided (e.g., insulin). In current commercially available devices, this marking procedure must be initiated by the user.

It should be noted that another deficiency of current commercially available meters is that the available marking options are is imprecise. In particular, a person with diabetes will experience rises in glucose levels following the consumption of food. For a health care provider to make appropriate adjustments in treatments, they need to understand if particular results came before or following meals (pre or post-prandial). While there are some devices that allow for marking of results directly on the analyte monitor, it is also quite common to use a paper “logbook” to track individual results. However, it is not sufficient to only associate a particular result with a particular meal. Instead, in order to more accurately interpret and utilize the test results, it should be associated with a time period before or after a particular meal.

Additional problems with current technology include:
patient compliance-many patients find marking meals confusing and simply choose not to mark any meals, or worse, will mark, perhaps without even realizing it, individual results as being associated with a particular meal when in fact the test did not actually occur before or after a meal.
lack of data, or inaccurate data, can lead to less than optimal treatment plans, or even worse risk of harm to the patient if too much or too little treatment (drugs, insulin etc.) are provided.
data accuracy—it is quite easy to make mistakes in marking meals using currently commercially available meters.
the process for marking directly on the device is not intuitive, users must initiate the marking process, which typically involves the need to memorize a long system of key/button presses or a need to consult their users guide for directions.
when using paper and pencil a user can transcribe the result incorrectly, or write the result in the wrong section of the logbook. Also, such information cannot be easily transferred or shared with health care professionals.
marking a test as only generally being associated with a particular meal does not provide fully accurate, useful information.
time-people with diabetes can spend a significant amount of time searching for logbooks, or re-reading instructions to understand how to mark meals.
safety-incomplete or inaccurate data can lead to mistakes in treatment harming patients.

While certain aspects of conventional technologies have been discussed to facilitate disclosure of the invention, Applicants in no way disclaim these technical aspects, and it is contemplated that the claimed invention may encompass or include one or more of the conventional technical aspects discussed herein.

### SUMMARY OF THE INVENTION

As used herein, “body fluid” encompasses whole blood, interstitial fluid, and mixtures thereof.

As used herein “integrated device” or “integrated meter” means a device or meter that includes all components necessary to perform sampling of body fluid, transport of body fluid, quantification of an analyte, and display of the amount of analyte contained in the sample of body fluid. Exemplary integrated meters are described in: U.S. Pat. Nos. 6,540,675 and 7,004,928; U.S. Patent Application Publication Nos. US 2008/0077048, US 2007/0179404, US 2007/0083131, US 2007/0179405, US 2007/0078358, and US 2007/0078313. The entire contents of each of the above-listed documents are incorporated herein by reference.

It is to be understood that reference herein to first, second, third and fourth components (etc.) does not limit the present invention to embodiments where each of these components is physically separable from one another. For example, a single physical element of the invention may perform the functions of more than one of the claimed first, second, third or fourth components. Conversely, a plurality of separate physical elements working together may perform the functions of one of the claimed first, second, third or fourth components. Similarly, reference to first, second (etc.) method steps does not limit the invention to only separate steps. According to the invention, a single method step may satisfy multiple steps described herein. Conversely, a plurality of method steps could, in combination, constitute a single method step recited herein. In addition, the steps of the method are not necessarily limited to the order in which they are described or claimed herein.

The present invention may address one or more of the problems and deficiencies of the prior art discussed above. However, it is contemplated that the invention may prove useful in addressing other problems and deficiencies, or provide benefits and advantages, in a number of technical areas. Therefore the claimed invention should not necessarily be construed as being limited to addressing any of the particular problems or deficiencies discussed herein.

The invention can be useful with any device, but is particularly applicable to analyte monitors used in a home or clinical setting such as glucose monitors. This invention is advantageous when used in conjunction with a fully integrated glucose meter. However, the invention is not so limited. The benefits and advantages of the invention can also be applied to other devices such as conventional (non-integrated) glucose meters and other self-diagnostic devices where collection of long term data and analysis of trends in data is important.

The present invention can provide one or more of the following benefits and advantages relative to current technology:
convenience of never having to look for instructions or paper log books, and the meter initiates the marking procedure, not the user. For example, according to the present invention, the results of the test can be displayed along with a reminder to mark the test as before or after a particular meal.
“screen real-estate”-size is a key factor for patients choosing a handheld analyte monitor and many of the LCD's used in these devices are created with fixed segments that are turned on or off to display relevant information. By compactly displaying all possible meal markers this invention saves space on the screen, which in turn allows for more of the screen used for other purposes and can therefore help reduce the overall size of the device.
significant increase in caregiver's confidence in data collected by the meter.
significant increase in the amount of accurate and useful data collected by user (pre and post prandial data).
improved ability to monitor/detect trends in test results.
ability to confidently adjust patient medications based on data collected by meter.
allows the meter to internally process data not available to currently used glucose monitors (e.g., a 7-day pre and/or post-meal average calculated by the meter).
accurate on-device averages of pre/post prandial results.
data once calculated by the device can easily be exported by the data management software used by healthcare professionals to pull data from patients' devices.
time/cost savings—by having the data automatically and accurately exported healthcare professionals are able to assist patients more quickly.
can provide users with information not available on conventional glucose meters.

According to one aspect, the present invention provides a method of monitoring the concentration of at least one target analyte in a sample of body fluid using a meter, the meter comprising a user interface, the method comprising:

obtaining a sample of body fluid; testing the sample to determine the concentration of the at least one target analyte contained therein; and presenting the user with a reminder to associate the test with an appropriate time corresponding to before or after a particular meal using the user interface.

According to a further aspect, the present invention provides a testing device comprising: a user interface, a processor and a memory, the device constructed an arranged to provide the results of a test and substantially simultaneously provide the user with a reminder to associate the results of the test with an appropriate time before or after a particular meal, and to store the associated results in the memory.

### BRIEF DESCRIPTION OF THE DRAWING FIGURES

The following description of preferred embodiments can be read in connection with the accompanying drawings in which like numerals designate like elements and in which:

FIG. 1 is a schematic plan view of certain embodiments of the present invention.

FIG. 2 is a partial plan view of certain alternative embodiments of the present invention.

FIG. 3 is an exemplary icon used in connection with certain aspects of the present invention, designating a first marked testing time (pre-breakfast).

FIG. 4 is the icon of FIG. 3, designating a second marked testing time (post-breakfast).

FIG. 5 is an illustration of a method or technique of the present invention, optionally Used in conjunction with the device of FIG. 2.

### DETAILED DESCRIPTION

It should be emphasized that the devices and methods described are intended to apply to any number of devices, meters or monitors. Thus, while according to certain embodiments, the principles of the present invention are applied to and used in conjunction with an integrated meter, the present invention is also usable with other devices such as conventional (non-integrated) analyte monitors. How the analyte measurement result is acquired by a particular device is not critical to implementation or practice of the present invention. The present invention is more relevant to what the device can do with results once collected. Thus, unless specifically stated to the contrary, the following description should be read as being applicable to any device, such as conventional non-integrated monitoring devices and systems, as well as integrated glucose monitors or meters. For example, when images of a display are shown or described the display could be that of any suitable device, such as a stand alone, test-strip-based device or a semi or fully integrated device. Also, the icons disclosed herein are associated with exemplary embodiments and may be changed, and still fall within the scope of the invention.

The invention provides, inter alia, an elegant and simple user interface that allows users of an analyte monitor to quickly and accurately associate a particular measurement as either pre or post-meal (pre/post-prandial).

One aspect of the invention is the functionality of a user interface that displays all possible meal time markings on a single portion of the display. Specifically, according to certain embodiments, all 6 possible meal markings are shown on the screen at one time (before or after each of breakfast, lunch, and dinner), with only one appropriate meal marker time ultimately selected. Users of the device can optionally cycle through all possible markings by interaction with an a user interface, for example, by pressing a simple up or down arrow on the side of the device or by utilizing a touch screen-type interface. Once the particular meal marker is suggested users can confirm their selection, also through interaction with the user interface, such as by pressing the power button which simultaneously marks a result and places the device into sleep mode, and/or by utilizing a touch screen-type interface.

Other aspects of the present invention involves methods or techniques for monitoring the concentration of at least one target analyte contained in a sample of body fluid. Any suitable target analyte may be monitored, such as glucose, hemoglobin, bilirubin, etc., or combinations thereof. Moreover, any suitable body fluid may be analyzed, such as saliva, urine, blood, interstitial fluid, or mixtures thereof.

As an initial step, a sample of body fluid is collected. Any suitable technique for the collection of body fluid is contemplated. For example, when the body fluid to be analyzed comprises blood, a sample can be obtained in a number of different ways. When the principles of the present invention are applied in the context of blood glucose monitoring, a sample of blood can be obtained, for example, by lancing a surface of the skin, thereby creating a wound from which a sample of blood can be obtained. Any suitable instrument can be used to create the wound, such as a solid lancet or hollow needle.

Subsequently, or concurrently, the sample is optionally transported to an appropriate analysis site, and analyzed to determine the concentration of the at least one target analyte contained therein. Any suitable technique for determining the concentration can be utilized. For example, when the principles of the present invention are utilized in the context of blood glucose monitoring, conventional electrochemical or colorimetric techniques can be utilized to ascertain the concentration of glucose contained in the sample of body fluid or blood. The results of the analysis are then presented to the user or tester. The results can be presented in any suitable manner, such as by visually displaying the results, and/or by audibly communicating the results.

According to the present invention, the user is also presented with a reminder to associate the results of the test with an appropriate time corresponding to before or after a particular meal. According to one alternative embodiment, this reminder is presented at approximately the same time as the results of the test are presented to the user. However, it should be recognized that this reminder can be provided at any suitable time so long as the user is reminded in adequate time to mark the results of the test in the desired fashion. This reminder can be presented to the user in any suitable manner. For instance, the reminder may be visually presented on a display, and/or by audibly communicating a reminder. When the reminder is visually presented on a display, any suitable symbol or combination of symbols can be utilized to communicate to the user that they should associate the test results with an appropriate time before or after a particular meal. According to one optional embodiment, an icon is displayed which contains a combination of symbols representative of all desired possible marking times. According to a further optional embodiment, an icon is displayed comprising symbols corresponding to breakfast (e.g., rising sun), lunch (e.g., midday sun), and dinner (e.g., moon), and the icon further comprises a selectable portion which can be selectively associated with an appropriate time before or after one of the above-mentioned meals. The selectable portion of the icon can be associated with an appropriate time by any suitable manner. Thus, according to certain optional embodiments, the selectable portion can be located appropriately by use of an interface device, such as one or more buttons, touch pads, touch screen, joysticks, and the like. Optionally, the selectable portion of the icon can be associated by using voice or audible commands in conjunction with voice/audible command recognition capabilities.

Further alternative embodiments include suggesting to the user an appropriate marking time for the just-completed test, based upon the time of day at which the test has been taken. For example, if the user completes a test at 6:30 AM it may be automatically suggested to the user that the test be marked as pre-breakfast. The automatic suggestion is again based upon the time of day, and optionally upon additional input which is preprogrammed and/or provided by the user. Thus, a device or mechanism can be provided which is preprogrammed to suggest that any test performed at 6:30 AM or earlier be suggested for marking as a pre-breakfast test. Alternatively, or in addition thereto, the user may customize this suggestion. For example, the user can specify that any test performed prior to 7 AM be suggested for marking as a pre-breakfast testing event.

Once the results of a particular test have been associated with an appropriate time before or after a particular meal, both the results of the test and the specified time association information is stored in any suitable manner using any suitable media. For example, the information can be stored as binary information in a memory device. According to certain embodiments, the information is stored in a format that is easily retrieved, shared and analyzed.

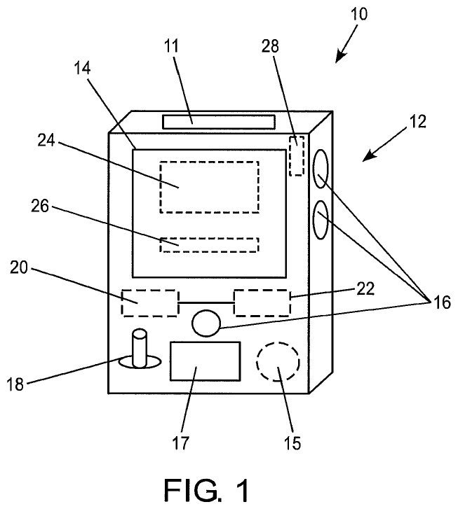

Further aspects of the present invention involves devices, systems, arrangements and the like which embody any or all of the above-mentioned functionality. Illustrative embodiments of such devices, systems and arrangements are described herein in connection with reference to FIGS. 1-5.

FIG. 1 illustrates an exemplary device 10. The device 10 is capable of determining the concentration of at least one target analyte contained in a sample of body fluid. Any suitable target analyte may be monitored, such as glucose, hemoglobin, bilirubin, etc., or combinations thereof. Moreover, any suitable body fluid may be analyzed, such as saliva, urine, blood, interstitial fluid, or mixtures thereof. As previously mentioned herein, such devices 10 can take a number of different forms. Thus, the device 10 can comprise an analyte monitor or meter that is designed to be used in conjunction with non-integrated systems. Thus, for example, the device 10 can be configured to cooperate with one or more of a separate test strip(s) and/or separate body fluid production/collection device(s). When configured in this manner, the device 10 can optionally include a slot or opening 11 which is configured to receive a separate test strip or cartridge therein, as previously mentioned above. When the device 10 is in the form of an integrated meter or monitor, it includes mechanisms for obtaining a sample of body fluid, analyzing the sample of body fluid, and presenting the results of the analysis, all within a single self-contained unit. Suitable integrated devices are mentioned herein, and the specifics of such constructions and modes of operation are described in the documents incorporated by reference herein.

The illustrated device 10 may include a suitable user interface 12. Alternatively, the interface can be provided separate from the device 10. For example, the device 10 may include all features necessary for analyzing a sample to determine the concentration of a target analyte contained therein, and be connected with a wired or wireless connection to a separate or remote interface, such as a display. The user interface 12 can include a display 14. Any suitable display is contemplated. According to one optional embodiment, the display 14 comprises an LCD. The user interface 12 may additionally include further components or features for interacting with a user. Thus, the user interface 12 may optionally include an audible input/output device 15, which may be in the form of a speaker and/or microphone. Additional components or features of the user interface 12 may optionally include input devices such as one or more of: buttons 16, touch pad 17, joystick 18, or any combination thereof. According to another optional construction encompassed by the above-described interface and display, the display 14 may also comprise a touch screen-type interface. According to further alternative aspects, the user interface 12 of the device 10 can include audible/voice recognition capabilities. For example, a user can interact with the device 10 by speaking or providing audible input via the audible input/output device 15, which are then interpreted and converted by the device into executable commands.

The device 10 may further be provided with a memory component 20 and a processor 22, which are operatively interconnected. Such components can be used according to generally known techniques to control the storage, manipulation and/or retrieval of information, as well as controlling, and responding to, the various components of the user interface 12.

The device 10 may include one or more sample transport features and/or analysis site comprising mechanisms for determining the concentration of at least one target analyte contained in the sample. For example, when the principles of the present invention are utilized in the context of blood glucose monitoring, conventional electrochemical or colorimetric mechanism can be included in the device 10 to ascertain the concentration of glucose contained in the sample of body fluid or blood. Examples of such mechanisms are described in greater detail in the documents incorporated herein by reference, and as previously pointed out, are not critical to practice the concepts of the present invention. The device 10 then presents the results of the analysis to the user. The results can be presented in any suitable manner, such as by visually displaying the results 24 on the display 14, and/or by audibly communicating the results via the audible input/output device 15.

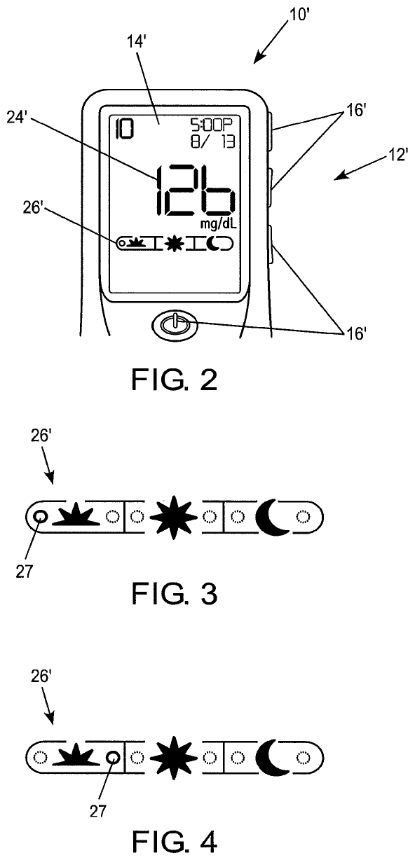

According to the present invention, the device 10 also presents the user with a reminder to associate the results of the test with an appropriate time corresponding to before or after a particular meal. According to one alternative embodiment, this reminder is presented at approximately the same time as the results of the test are presented to the user. However, it should be recognized that this reminder can be provided at any suitable time so long as the user is reminded in adequate time to mark the results of the test in the desired fashion. This reminder can be presented to the user in any suitable manner. For instance, the reminder 26 may be visually presented on the display 14, and/or by audibly communicating a reminder via the audible input/output device 15. When the reminder 26 is visually presented on a display 14, any suitable symbol or combination of symbols can be utilized to communicate to the user that they should associate the test results with an appropriate time before or after a particular meal. According to one optional embodiment, the reminder 26 comprises an icon that is displayed which contains a combination of symbols representative of all desired possible marking times. According to a further optional embodiment, the reminder 26 comprises an icon is having symbols corresponding to breakfast (e.g., rising sun), lunch (e.g., midday sun), and dinner (e.g., moon), and the icon further comprises a selectable portion (e.g., 26′, FIGS. 2-5) which can be selectively associated with an appropriate time for or after one of the above-mentioned meals. The selectable portion of the icon can be associated with an appropriate time by any suitable manner. Thus, according to certain optional embodiment, the selectable portion can be located appropriately by use of an interface device, such as one or more buttons 16, touch pads 17, touch screens 14, joysticks 18, and the like. Optionally, the selectable portion of the icon can be associated by using voice or audible commands via audible input/output device 15, in conjunction with voice/audible recognition capabilities possessed by the device 10.

The device 10 may further include the capability to suggest to the user an appropriate marking time for the just-completed test, based upon the time of day at which the test has been taken. For example, the device 10 is provided with a clock 28. The clock 28 can be manually set by the user, or may be automatically set and/or adjusted by any suitable mechanism. Such a device incorporating an automatic clock is disclosed in US 2010-0021948, the entire contents of which is incorporated herein by reference. In either case, if the user completes a test at 6:30 AM, the device 10 may suggest to the user via the user interface 12, in any manner described herein, to mark the test as pre-breakfast. The automatic suggestion is again based upon the time of day, and optionally upon additional input which is preprogrammed or provided by the user. Thus, the device 10 can be preprogrammed (e.g., factory programmed) to suggest that any test performed at 6:30 AM or earlier be suggested for marking as a pre-breakfast test. Alternatively, or in addition thereto, the user may customize this suggestion. For example, the user can program the device 10 to specify that any test performed prior to 7 AM be suggested for marking as a pre-breakfast testing event.

Once the results of a particular test have been associated with an appropriate time before or after a particular meal, both the results of the test and the specified time association information is stored in any suitable manner using any suitable media, such as the memory 20. For example, the information can be stored as binary information in the memory device 20. According to certain embodiments, the information is stored in a format that is easily retrieved, shared and analyzed.

A device constructed according to further alternative embodiments, and associated methods, are illustrated and described in connection with FIGS. 2-5. It should be understood that this embodiment may include any or all of the previously described functionality and/or features of the previously described embodiments.

The device 10′ illustrated therein can be in the form of an integrated monitor or meter. Thus, it may possess any or all of the features associated with such integrated monitors, and as described in the documents incorporated by reference herein. In addition, the previously described principles of the present invention, when applied to such an integrated device 10′, possesses numerous benefits and advantages, as generally described herein.

As with the previously described embodiments, the device 10′ optionally includes a user interface 12′ associated with the device 10′. The user interface 12′ can include a number of features, such as a display 14′, which may also comprise a touch-screen-type interface, and/or one or more buttons 16′. Upon analysis of a suitable sample of body fluid, the device 10′ analyzes the sample and determines the concentration of at least one target analyte contained therein. These results are then presented to the user. According to the illustrated embodiment, the results 24′ are presented to the user by displaying them on the display 14′. The electronics, and interactions therewith necessary to accomplish this display functionality is generally well-known to those in the art, and is not critical practice the principles of the present invention. In addition, the device 10′ presents a reminder to the user to associate the results 24′ with an appropriate time before or after a particular meal. The device 10′ can present this reminder in any suitable manner. According to the illustrated embodiment, the reminder is presented on the display 14′ in the form of an icon 26′. The icon 26′ can comprise any suitable symbol or combination of symbols to communicate to the user that they should associate the test results with an appropriate time before or after a particular meal. According to the illustrated embodiment, the icon 26′ contains a combination of symbols representative of all desired possible marking times. Further, the illustrated embodiment comprises symbols corresponding to breakfast (e.g., rising sun), lunch (e.g., midday sun), and dinner (e.g., moon), and the icon 26′ further comprises a selectable portion 27 which can be selectively associated with an appropriate time before or after one of the above-mentioned meals. The selectable portion 27 of the icon can be associated with an appropriate time/meal by any suitable manner. Thus, according to certain optional embodiments, the selectable portion 27 can be located appropriately by use of an interface device, such as one or more buttons 16′, or a touch screen 14′. As with the previously described embodiments, the selectable portion 27 can be manipulated with alternative interface devices such as, touch pads, touch screens, joysticks, and audible commands, which may optionally form part of the device 10′.

The device 10′ may optionally further include the previously described features and functionality associated with suggesting an appropriate time marking to the user based upon the time of day at which the test is conducted. The device 10′ may optionally utilize a manually set or automated clock, as previously described.

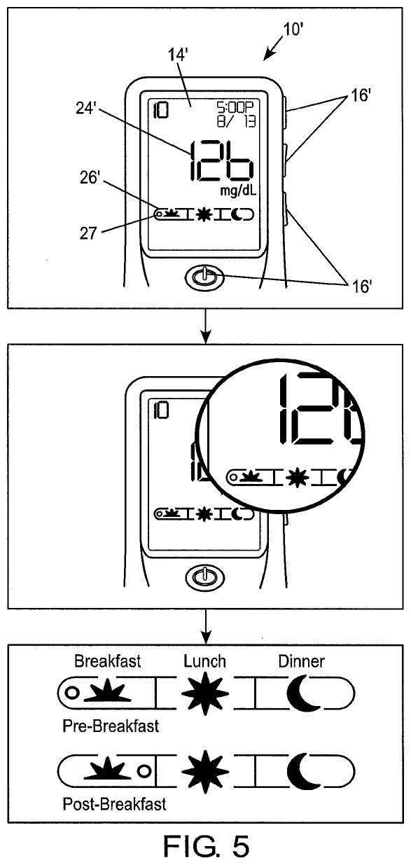

One possible mode of operation of the device 10′ is illustrated in FIG. 5. The integrated device 10′ is utilized in a known manner to produce and collect a sample of body fluid, transport the sample to an appropriate analysis site within the device 10′, and analyzed a sample to determine the concentration of a target analyte contained therein. Once this analysis has been performed, the results 24′ are presented to the user on the display 14′. According to the illustrated embodiment, a reminder to mark the results as associated with an appropriate time before or after a particular meal is also displayed or presented to the user at substantially the same time as the display of the results 24′. Although it is contemplated that the reminder may be presented at a time which is before or after presentation of the results, as previously noted herein. According to the illustrated embodiment, the reminder is in the form of an icon 26′. The icon 26′ contains a combination of symbols representing breakfast, lunch and dinner. The icon 26′ further includes a selectable portion 27 for associating the results of the test with a time before or after a particular meal. The selectable portion 27 is optionally suggested to the user based at least in part upon the time of day at which the test is conducted as indicated by the clock 28 of the device 10′. According to one alternative, the selectable portion 27 flashes in the suggested location. Regardless of whether an appropriate marking is suggested by the device 10′, the user may change the position of the selected portion by using any suitable interface, such as the buttons 16′ of the device 10′, or by a touch screen 14′. Thus, according to the illustrated embodiment, should the user agree with the suggested marking, the user can make the selection by any suitable means, such as by simply pressing the power button to confirm that this is the appropriate time marking for the testing event. If the user wishes to deviate from the suggested marking position, or independently select an appropriate marking position for the selectable portion 27 of the icon 26′, the user can change the position of the selectable portion 27 by use of one or more of the buttons 16′ of the device 10′, or by utilizing a touch screen 14′. Again, once the user is satisfied with the appropriate positioning of the selectable portion 27, the choice is confirmed (e.g., by pushing the power button), and the procedure is completed. The device 10′ then stores the results along with the associated time information in a memory 20 of the device 10′, which can then be retrieved and analyzed either with by the device itself 10′, or by external devices (not shown).

Numbers expressing quantities of ingredients, constituents, reaction conditions, and so forth used in this specification are to be understood as being modified in all instances by the term “about”. Notwithstanding that the numerical ranges and parameters setting forth, the broad scope of the subject matter presented herein are approximations, the numerical values set forth are indicated as precisely as possible. Any numerical value, however, may inherently contain certain errors necessarily resulting from the standard deviation found in their respective measurement techniques. None of the elements recited in the appended claims should be interpreted as invoking 35 U.S.C. § 112, 116, unless the term “means” is explicitly used.

Although the present invention has been described in connection with preferred embodiments thereof, it will be appreciated by those skilled in the art that additions, deletions, modifications, and substitutions not specifically described may be made without departing from the spirit and scope of the invention as defined in the appended claims.

1. A method of monitoring the concentration of at least one target analyte in a sample of body fluid using a meter, the meter comprising a user interface, the method comprising:
obtaining a sample of body fluid;
testing the sample to determine the concentration of the at least one target analyte contained therein; and
presenting the user with a reminder to associate the test with an appropriate time corresponding to before or after a particular meal using the user interface.

2. The method of claim 1, wherein the target analyte comprises glucose, and the body fluid comprises blood.

3. The method of claim 1, wherein the meter comprises an integrated device, the integrated device constructed and arranged to collect the sample of body fluid from the user, test the sample to determine the concentration of target analyte contained therein, and present the results of the test to the user, all in a single unitary device.

4. (canceled)

5. (canceled)

6. The method of claim 1, wherein obtaining a sample of body fluid comprises piercing the skin of the user thereby creating a wound, and obtaining the sample of body fluid from the wound.

7. (canceled)

8. (canceled)

9. The method of claim 1, wherein presenting the user with a reminder comprises displaying an icon on the user interface.

10. The method of claim 1, wherein presenting the user with a reminder comprises producing an audible signal.

11. The method of claim 9, the icon representing all appropriate times that may be selected by the user.

12. The method of claim 9, wherein the icon comprises at least one symbol corresponding to a particular meal.

13. The method of claim 9, wherein the icon comprises symbols that correspond to breakfast, lunch, and dinner.

14. (canceled)

15. (canceled)

16. The method of claim 1, wherein the user interface comprises a display, and the method further comprises displaying the concentration of the target analyte determined by the meter, and substantially simultaneously displaying the reminder to associate the test with an appropriate time corresponding to before or after a particular meal.

17. (canceled)

18. (canceled)

19. The method of claim 1, wherein the concentration of the at least one target analyte and the association thereof with an appropriate time comprises data, and wherein the meter comprises a memory, and the method further comprising storing the data in the memory.

20-27. (canceled)

28. A testing device comprising: a user interface, a processor and a memory, the device constructed an arranged to provide the results of a test and substantially simultaneously provide the user with a reminder to associate the results of the test with an appropriate time before or after a particular meal, and to store the associated results in the memory.

29. The device of claim 28, wherein the user interface comprises a display.

30. (canceled)

31. The device of claim 28, wherein the testing device comprises a meter constructed to determine the concentration of at least one target analyte contained in a sample of body fluid.

32. The device of claim 31, wherein the analyte comprises glucose and the body fluid comprises blood.

33. The device of claim 32, wherein the meter comprises an integrated meter, the integrated meter constructed and arranged to collect the sample of body fluid from the user, test the sample to determine the concentration of target analyte contained therein, and present the results of the test to the user, all in a single unitary device.

34. The device of claim 33, the device further comprising an analysis site and a mechanism for transporting the sample of body fluid to the analysis site within the meter.

35. The device of claim 34, further comprising a mechanism for analyzing the sample using a colorimetric technique to determine the concentration of the target analyte contained therein.

36. The device of claim 35, wherein the user interface comprises a display, and device is constructed to display an icon on the user interface as a reminder to associate the results of the test with an appropriate time before or after a particular meal, and wherein the device is further configured to store the associated results in the memory.

37-44. (canceled)

45. A user interface comprising a display configured to present the user with a reminder to associate the test with an appropriate time corresponding to before or after a particular meal.
